(19) United States
(12) Patent Application Publication
(10) Pub. No.: US 2010/0256067 A1
(43) Pub. Date: Oct. 7, 2010

(54) LIQUID EDIBLE COMPOSITION COMPRISING ISOFLAVONES

(76) Inventors: Yvonne Evelien Bruggeman, Vlaardingen (NL); Demet Güzey, Vlaardingen (NL); Nicolette Petronella M. Straatjes, Vlaardingen (NL)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/738,190

(22) PCT Filed: Oct. 8, 2008
(86) PCT No.: PCT/EP08/63464

§ 371 Date: Jun. 16, 2010

(30) Foreign Application Priority Data
Oct. 18, 2007 (EP) 07118805.6

(51) Int. Cl. A61K 8/64; A61Q 19/00

(52) U.S. Cl. 514/18.8

(57) ABSTRACT

A liquid edible product comprising isoflavones, water, and which further comprises β-lactoglobulin.

### FIELD OF THE INVENTION

The present invention relates to a liquid product comprising isoflavones, water, and which further comprises β-lactoglobulin, a process to prepare such, and the use of such product as a skin care by ingestion product.

### BACKGROUND OF THE INVENTION

A group of compounds that is reported to have beneficial effects, when ingested in the proper amounts, for certain aspects of health and/or appearance like skin condition are isoflavones. For such effects, a daily intake of 10-200 mg per day is believed to be effective. These small amounts can be offered to consumers as a health supplement in the form of e.g. pills or capsules, together with other materials like minerals, vitamins, and so on. However, there is a demand also for a method to supply such amounts of isoflavones as a drinkable product. Although isoflavones occur naturally in e.g. soy milks, the level of these is generally too low to be effective, or unnatural high amounts of soy milk would need to be ingested. Hence, there is a need for a liquid edible composition which contains a certain level of isoflavones, which level is such that only a limited amount of such liquid needs to be consumed to obtain the daily intake above referred. In particular, there is a desire for such liquid product which contains such isoflavones in a concentration that only a limited volume needs to be consumed, e.g. a volume of between 25-250 ml, preferably 30-150 ml, more preferably 40-100 ml.

A problem, however, with wishing to offer isoflavones in liquid products is that isoflavones are not soluble in water or (triglyceride) oil: that is, not in the concentrations desired. Hence, when isoflavones are added to water, even when well dispersed e.g. by homogenisation techniques, they tend to settle and form a sediment over time (e.g. over weeks or months storage time). Although not harmful, the consumer, when wishing to consume such product, runs the risk that not all isoflavones which were put in the product are consumed, as some may be left behind in the packaging, and under dosing may occur. Also, the settling isoflavones may take other components with them in settling. Of course a solution may be to give the consumer the instruction to shake the packaging of liquid before opening, but such does not solve all the problems, as it has been found that such sediments can be formed in such a way that a normal shaking of the container (e.g. a bottle) by hand is not sufficient to redisperse the majority of the isoflavones. Apparently the isoflavones may form a compact sediment, too compact after prolonged storage to redisperse by shaking by hand in reasonable time and effort.

### SUMMARY OF THE INVENTION

Hence, there is a need for a way to include isoflavones in a liquid edible product, in such a way that sedimentation is reduced, or, if sedimentation still occurs, that the sediment is of such nature that it can be redispersed again (after e.g. 1 month storage at ambient or chilled temperature), at least in a major extent, i.e. at least 50%, or that the sediment which remains after shaking gives the impression that only a minor amount of sediment remains on the bottom of the container after shaking the container, compared to what was put in the liquid. Shaking is herein to be understood as shaking by hand, especially of a container of 1000 ml or less, down to 20 ml, with e.g. 10 shaking movements (1 shaking movement herein defined as shaking by hand, one time back and forth roughly along the central axis of the bottle or other packaging, as is usually done by consumers wishing to shake e.g. a 350 ml bottle by a quick “pumping” movement of the underarm). Preferably, the composition should be such that it allows e.g. 10-400 mg of isoflavones in one serving of liquid, one serving preferably being 25-250 ml, more preferably 30-150 ml, and even more preferably 40-100 ml liquid.

Consumers can also be interested in multi-component health foods. Also, there is a range of components to which improved health and/or appearance have been attributed upon consumption. Examples of such components are vitamins like vitamins C, E and several of the B's, but also components like carotenoids and omega-3 fatty acids and/or omega-6 fatty acids and/or esters thereof. Some of these components are water soluble (e.g. vitamins B and C, and some of the carotenoids, depending on their origin and/or carrier), and some are fat soluble, like other carotenoids, vitamin E and omega-3 fatty acids and/or omega-6 fatty acids and/or esters thereof. Hence, there is also a desire that the liquid edible product comprising isoflavones can also comprise one or more of these additional actives. Regarding omega-3 fatty acids and/or omega-6 fatty acids and/or esters thereof, they preferably should be incorporable in such liquid edible product in an amount of between 0.2 and 5 g, for the amount of liquid given, in a concentration of 0.05-10%, preferably 0.08-3% (wt).

Hence, there is a need for a liquid edible product comprising isoflavones (preferably present in said liquid edible composition in an amount of 0.005-0.5%, by weight, based on the total formulation, more preferably 0.01-0.2%, and even more preferably 0.02-0.1%, by weight based on the total composition, and most preferably 0.03-0.1%) which product further comprises water, and which product, after being stored for 1 month at chilled conditions (5° C.) may form a sediment, but which sediment (if occurring) may be redispersed by shaking an appropriate container by hand, for e.g. 10 shaking movements, such that at least 50%, preferably at least 80%, more preferably at least 90% (by weight) of the isoflavones present in the product is redispersed, preferably homogeneously, in the liquid, to remain there for at least 3 minutes to allow consumption. In the alternative, or preferably jointly achieved target, there is desired a product of the same composition, in which the sediment of isoflavone may be redispersed in the same manner and to the same standard, but after 1 month storage at ambient conditions (e.g. about 25° C.). Preferably, such is achieved for one or both temperatures after 6 weeks storage, or more preferably for one or both temperatures after 3 months storage.

It may be further preferred that such product allows the inclusion of one or more of the other active components referred to above, in amounts in which they are believed to be beneficial for health and/or appearance and/or skin condition.

Preferably, the product should be easily drinkable from e.g. a small bottle, of e.g. 25-250 ml, preferably 30-150 ml, more preferably 40-100 ml size, after shaking by hand for e.g. 15 seconds, preferably 12 seconds. Preferably the product should be liquid at chilled temperatures (e.g. 5° C.). The desire to be liquid is in part for reasons of consumer preference, but to a large extent determined by the small volume desired in combination with effective dosing: if e.g. a volume of e.g. 100 ml is to be consumed, then if the product is too viscous, a relative large proportion of the product will remain in the container, resulting in possible consumer disliking and/or ingestion of an incomplete dosing of the actives. Hence, it is preferred that the product should have a viscosity of 0.005-0.2 Pa·s, when measured at a shear rate of 10 (s⁻¹) at 7° C., using the following equipment: Advanced Rheometer AR 1000, spindle: conic, 2°0′38″, diameter (mm) 60, truncation (microm) 59. Preferably, said viscosity is 0.01-0.15 Pa·s, more preferably 0.01-0.1 Pa·s. Measuring at a shear rate of 10 (s⁻¹) is believed to be representative of pouring/drinking out of a small (e.g. 60 ml) bottle.

Preferably, such composition should be easy to manufacture, and be suitable to heat pasteurisation or sterilisation. Preferably, the product has (when packaged) a low pH, for reasons of preservation (e.g. for pasteurised products) and/or for reasons of taste, as such low pH products can be easily flavoured with a fruit-like flavour which is a flavour liked by large groups of consumers, especially the targeted group of consumers (e.g. adult women, or mature women). Hence, a pH of e.g. below 5 is preferred, more preferably a pH of 2.8-4.5, more preferably 3-4.

It has now been found that the above formulated objectives can be met (at least in part) by a liquid composition comprising:
isoflavones in an amount of 0.005-0.5%, by weight, based on the total formulation,
water in an amount of 40-98% (by weight, based on the total formulation),
β-lactoglobulin in an amount of 0.1-2% (by weight on the total formulation)
   wherein the composition comprises casein in amount less than 0.5%, by weight based on the total composition, and wherein the weight ratio β-lactoglobulin to isoflavone is between 2 and 30.

The invention further relates to a process for preparing a liquid composition comprising isoflavones in an amount of 0.005-0.5% (by weight, based on the total formulation), water in an amount of 40-98% (by weight, based on the total formulation), and which further comprises β-lactoglobulin in an amount of 0.1-2% (by weight on the total formulation), and casein in amount less than 0.5%, by weight based on the total composition, and wherein the weight ratio β-lactoglobulin to isoflavone is between 2 and 30, which process comprises the steps of:
a) preparing a mixture comprising the water, isoflavones and β-lactoglobulin,
b) vigorous stirring said mixture,
c) subjecting to a pasteurising or sterilising treatment and packing, in any given order.
   Vigorous stirring, however, may also be carried out after pasteurising or sterilising treatment (and before packaging).

### DETAILED DESCRIPTION OF THE INVENTION

In the present invention, the levels of β-lactoglobulin that are required or preferred are actually measured as amount of whey protein isolate needed, as such is a convenient source of β-lactoglobulin. The preferred amounts of whey protein isolate to be used in the compositions, process and use according to the present invention are (in weight % based on the total composition): from 0.15% to 3%, preferably from 0.15% to 1.5%, more preferably from 0.23% to 0.75%, most preferably from 0.38% to 0.68%. When assuming a total level of protein of 90% in whey protein isolate, and a level of β-lactoglobulin of 75% of the protein fraction of whey protein isolate this gives the required or preferred levels of β-lactoglobulin.

Isoflavones in the present invention preferably are compounds having a 3-phenyl-4H-1-benzopyr-4-one backbone, and more preferably such comprises isoflavones (including aglycon forms) and/or derivates thereof, like β-glucoside-, acetyl- and malonyl- methylated- and glycoside-forms. References describing such are e.g. Song et al. 1999. Journal of Agricultural and Food Chemistry 47: 1607-1610; Kudou et al. 1991. Agricultural and Biological Chemistry 55:2227-2233; Wang et al. 1998. Journal of the American Oil Chemists Society 75: 337-341; and Choi and Rhee. J. of Med Food 9 (1) 2006, 1-10. Isoflavones can e.g. be found in soy, clover sprouts and a number of legumes like chick peas, and products (including extracts and concentrates) derived from these. All the above forms are herein collectively referred to as “isoflavones”. Even more preferred isoflavones herein comprise formononetin, biochanin A, daidzein, genistein and glycitein (formononetin and biochanin A are the methylated forms of daidzein and genistein), daidzin, genistin, glycitin, and/or mixtures thereof.

The amount of isoflavone in the present composition may be 0.005-0.5%, by weight, based on the total formulation, but is preferably 0.01-0.2%, more preferably 0.02-0.1%, by weight based on the total composition, and most preferably 0.03-0.1%. The isoflavone particles herein have a size of around 5-15 micron.

It was surprisingly found that β-lactoglobulin, although it does not fully prevent sedimentation of the isoflavones, it makes the isoflavone apparently (at least for the first 6 weeks) in such a way that although they still may sediment, the sediment formed is apparently such, that it is comparatively easy to redisperse such sediment again, e.g. by shaking a container comprising the composition according to the invention by hand, as set out above in the objective and invention. Without wishing to be bound by theory, it was believed that the use of β-lactoglobulin makes the sediment more fluffy than without β-lactoglobulin, and it is believed (without wishing to be bound by theory) that it is this fluffiness that makes the isoflavones relatively easy redispersible, when used in accordance with the present invention.

It was also found that the product has a viscosity that allows easy pouring and/or drinking from a small container (e.g. 60 or 100 ml) and that also allows easy shaking to redisperse the isoflavones. Hence, it is preferred that the product according to the present invention has a viscosity of 0.005-0.2 Pa·s, when measured at a shear rate of 10 (s⁻¹) at 7° C., using the following equipment: Advanced Rheometer AR 1000, spindle: conic, 2°0′38″, diameter (mm) 60, truncation (microm) 59. Preferably, said viscosity is 0.01-0.15 Pa·s, more preferably 0.01-0.1 Pa·s. Measuring at a shear rate of 10 (s⁻¹) is believed to be representative of pouring/drinking out of a small (e.g. 60 ml) bottle.

Needless to say the container plus composition should be such that it allows mixing by shaking by hand, and thus it should have sufficient headspace in the container (either before or after opening) and/or a mixing device should be present in the container.

In the composition according to the invention, the amount of β-lactoglobulin in the composition is preferably 0.1-1%, more preferably 0.15-0.5% (by weight on the total formulation), and most preferably 0.25-0.45%. The more isoflavone is used, the higher the amount of β-lactoglobulin is desired. Hence, it is preferred that in the present composition the weight ratio β-lactoglobulin to isoflavone is between 2 and 15, more preferably between 3 and 10. When β-lactoglobulin is present as whey protein isolate, and wherein the β-lactoglobulin is calculated as whey protein isolate, it is preferred that the (weight) ratio whey protein isolate: isoflavone is preferably between 1.5 and 40, more preferably between 3 and 22, most preferably between 4.5 and 15.

The protein β-lactoglobulin is a component present in whey protein, and whey protein can be obtained from dairy proteins, which can be obtained from milk, which contains next to these proteins e.g. other proteins (e.g. casein) and lactose. Conventional dairy protein is a mixture comprising e.g. about 80% casein proteins, and about 20% whey protein. Whey protein is a mixture of several proteins, of which β-lactoglobulin is a major one, occurring in whey protein in an amount of about 75%.

Hence, a convenient way to use the β-lactoglobulin in the compositions according to the present invention is in the form of a whey protein composition, provided the amount of casein is below the claimed limit. Hence, it is preferred that the β-lactoglobulin in the compositions according to the invention originates from whey protein, a whey protein concentrate, or a whey protein isolate. One can add the β-lactoglobulin as such (i.e. as a purified compound) or use it in the form of the above referred whey protein compositions, as long as care is taken that sufficient whey protein composition is taken to have the desired β-lactoglobulin level, and as long as care is taken that the amount of casein is within the limits as set out herein. Most preferably, the β-lactoglobulin originates from whey protein isolate (WPI), as WPI is a protein mixture fairly high in β-lactoglobulin and yet still easily available. In view of this it is preferred that the composition according to this invention comprises whey protein isolate in an amount of 0.1-2%, preferably 0.2-1.5% by weight, based on the total composition, wherein the amount of casein in the composition is less than 0.5%, preferably less than 0.2%, more preferably less than 0.1% by weight, based on the total composition. Whey compositions like whey protein concentrates and whey protein isolates may contain part of their protein in hydrolysed form. It is preferred that a whey protein concentrate or whey protein isolate is used in the present invention which has a low level of hydrolysed proteins. Preferably, a whey protein isolate is used having a level of whey protein of at least 80%, preferably at least 85%.

It may also be preferred to keep the level of lactose in the present composition below a level of 0.3%, by weight based on the total composition. More preferably, said level is below 0.2%, and even more preferably said level is below 0.1%, and most preferred below 0.05%. Alternatively, the level of lactose is preferably less than 30% of the level of β-lactoglobulin in the composition, more preferably less than 20%, most preferably less than 10% of the level of β-lactoglobulin in the composition. Following this, the source of β-lactoglobulin in the present composition is preferably whey protein isolate, as such is still a widely available β-lactoglobulin source, yet fairly low in lactose. Levels of minerals in the composition should preferably be low.

It was found that in the compositions as set out herein, that the presence of a charged biopolymer may further improve the “redispersibility” of the isoflavones, in particular anionic charged biopolymers. Hence, it is preferred that the compositions according to the present invention further comprise a charged (preferably anionic) biopolymer in an amount of 0.1-1.2%, preferably an amount of 0.2-0.6% by weight, based on the total composition.

A charged biopolymer may help especially, but not exclusively if the composition has a pH of below 7, e.g. below 5, preferably 2.8-4.5, more preferably 3-4. Hence, preferably the product according to the invention has a pH of below 5, preferably 2.8-4.5, more preferably 3-4. Such pH may also be preferred for preservation purposes, and possibly for taste reasons.

Preferably, said charged biopolymer comprises a carbohydrate-based biopolymer. In this, pectin, and in particular high methoxylated pectin being most preferred, in particular when the liquid composition according to the present invention has a pH below the iso-electric point of β-lactoglobulin, which is about 4.7 When the composition is more neutral, e.g. at pH between 6 and 8, it may be preferred that the charged biopolymer comprises carrageenan.

It may be preferred that the total level of proteins is kept within limits. Needless to say β-lactoglobulin needs to be present, but it is preferred that the total level of proteins (including β-lactoglobulin) in the composition is less than twice the amount of β-lactoglobulin present. β-Lactoglobulin (or whey protein isolates and other whey preparations) occur frequently next to other diary proteins, like casein. In the present invention, it was found, however, that redispersibility of the isoflavones benefits from the condition that the amount of other dairy proteins, in particular casein, is low. Hence, in the compositions according to the invention, the ratio casein to β-lactoglobulin is below 2, preferably below 1. Formulated in an alternative way, the composition comprises casein in amount less than 0.5% by weight based on the total composition, preferably less than 0.2%, more preferably less than 0.1%. Most preferred is casein being substantially absent, but such is difficult to realise.

The compositions according to the present invention may further comprise additional ingredients of which it is believed or thought that they may provide health effects or (possibly) be (when ingested) beneficial for the health and/or appearance of skin, skin condition, skin feeling, and/or may contribute to reducing wrinkles in certain skin area's. Examples of such products are carotenoids like lycopene, and/or vitamin E, and/or vitamin C. When a carotenoid is present, such is preferably present in an amount of 0.5 mg-100 mg, more preferably 1-15 mg (when taken as pure carotenoid), or in a concentration in the liquid of the present invention of 0.001-0.05%, preferably 0.002-0.01%. When vitamin E is present, such is preferably present in an amount of 5 mg-1000 mg, more preferably 10-150 mg, or in a concentration in the liquid of the present invention of 0.01-0.5%, preferably 0.02-0.1%. When vitamin C is present, such is preferably present in an amount of 30 mg-2000 mg, more preferably 60-1000 mg, or in a concentration in the liquid of the present invention of 0.05-0.3%, preferably 0.1-0.5%.

In this, the carotenoid is preferably of vegetable origin, e.g. for reasons of food safety, labeling, permission and product image. Many of such carotenoids from vegetable origin, however, contain still traces (or more than traces) of the material from which they are obtained, and/or carrier materials as they are offered commercially. The presence of such material from which the carotenoids may be obtained (e.g. tomato fibre for lycopene) and/or carrier thereof may also have an effect on a sediment formed, and the ease of redispersing it.

When it is referred to herein as “vitamin C”, it is to be understood as to comprise also one or more of its salts and precursors, such as ascorbic acid and salts of ascorbic acid and/or sources thereof.

When it is referred to herein as “vitamin E”, it is to be understood as to comprise also one or more of various forms of vitamin E, like tocopheryl acetate, and more specifically d-α-tocopheryl acetate, as well as sources of these compounds.

When it is referred to herein as “carotenoids”, it is to be understood as to comprise one or more of lycopene, beta-carotene, lutein, astaxanthin, zeaxanthin, alpha-carotene, beta-cryptocanthin, canthaxanthin. It also covers sources of carotenoids, in particular when they contain at least 1% of such carotenoids. An example of this is a tomato fraction which is high in lycopene. It also covers carotenoids and/or sources thereof which are mixed with an auxiliary component, such as a carrier like maltodextrin.

The compositions according to the present invention may also further comprise other components, such as hydrophobic matter, as long as it remains a liquid with a water continuous phase. Hence, the composition may comprise 0.2-10% of a hydrophobic matter. Preferred hydrophobic matter herein is hydrophobic matter which comprises omega-3 fatty acids and/or omega-6 fatty acids and/or esters thereof, preferably in an amount of 0.4-5 g. The presence of these compounds is not so much preferred for preventing sedimentation, but as it is believed that the beneficial effect on health and/or appearance and/or skin condition (e.g. wrinkles) of isoflavones is further enhanced by the presence of such omega-3 fatty acids and/or omega-6 fatty acids and/or esters thereof. When such hydrophobic matter is included, the process to prepare such preferably includes a homogenisation step, in addition to and/or in stead of the vigorous stirring needed to disperse e.g. the isoflavones.

Regarding said omega-3 fatty acids and/or omega-6 fatty acids and/or esters thereof, said fatty acids preferably have 18-30 carbon atoms and contain at least three double bonds, more preferably 20-24 carbon atoms and contain at least three double bonds. These omega-3 fatty acids and/or omega-6 fatty acids can be in the form of free fatty acid, C1 to C6 alkyl esters thereof, glycerides (including mono-, di- and tri-glycerides, including di- and tri-glycerides wherein omega-3 fatty acids and/or omega-6 fatty acids are present in the di- or tri-glyceride next to other fatty acids which may not necessarily be omega-3 fatty acids and/or omega-6 fatty acids) thereof, phospholipid esters thereof or mixtures thereof, all of which are herein collectively referred to as “omega-3 fatty acids and/or omega-6 fatty acids and/or esters thereof”. Such omega-3 fatty acids and/or omega-6 fatty acids and/or esters thereof can be obtained e.g. from marine oils such as fish oils, fish liver oils, and algae, and some specific vegetable oils like borage. Preferred omega-3 fatty acids and/or omega-6 fatty acids (and their derivatives as set out above for the omega-3 fatty acids and/or omega-6 fatty acids) in the present invention comprise DHA (docosahexaenoic acid) and/or EPA (eicosapentaenoic acid), and/or esters thereof. It is also to be understood herein, that whenever an amount of omega-3 fatty acids and/or omega-6 fatty acids and/or esters thereof is referred to, such amount is to be calculated as if it were triacylglycerides.

The compositions according to the invention may comprise other ingredients, like e.g. one or more of: pH reducing agents, flavourings, carotenoids, vitamins, minerals, flavouring agents, colouring agents, vegetable oil.

The composition according to the present invention is a liquid composition. Liquid in this as easily flowable, such that it is e.g. easily drinkable or pourable from a bottle. Preferably the rheology should also be such that when the product is stored chilled, the liquid composition is pourable at 5° C. This means that the level of thickeners should be kept within limits. In fact, next to the charged biopolymer referred to, the present composition is preferably free of additional thickeners.

In the process to prepare the compositions according to this invention, which process is set out above, it is preferred that the β-lactoglobulin is added to (part of the) water first before being mixed with other ingredients. Preferably, the water has a temperature of 20-60° C., when the β-lactoglobulin is added to the aqueous phase. The amount of water needed for this can easily be determined by the person of average skill in the art, e.g. 10-100% of the total amount of water present in the composition.

To facilitate processing and/or dispersibility if the isoflavones, in the process according to the present invention, the isoflavones are preferably added to the other components being dispersed in a hydrophobic compound, preferably a triglyceride like a vegetable oil, in an amount which is between 1 and 100 times the amount of isoflavones, e.g. 0.2-2% by weight of the total composition. In the alternative, the isoflavones may also be added to a water fraction (e.g. part or all of the water of the composition) instead of oil, e.g. if no oil phase is present.

When further hydrophobic matter is to be included in the compositions, such as e.g. omega-3 fatty acids and/or omega-6 fatty acids and/or esters thereof, such is preferably done during any step prior to step c), and when such hydrophobic matter is included in the formulation, the vigorous stirring preferably comprises homogenisation. Also, prior to adding such omega-3 fatty acids and/or omega-6 fatty acids and/or esters thereof, the mixture to which it is added is preferably deaerated.

When other compounds like one or more of a charged biopolymer, pH reducing agents, flavourings, carotenoids, vitamins, minerals, flavouring agents, colouring agents, vegetable oil are to be included in the composition according to the invention, such is preferably achieved at any step before step c).

The present invention further relates to a process for preparing a liquid composition ready for consumption, said liquid composition comprising isoflavones in an amount of 0.005-0.5%, by weight, based on the total formulation, water in an amount of 40-98% (by weight, based on the total formulation), β-lactoglobulin in an amount of 0.1-2% (by weight on the total formulation), and casein in an amount of less than 0.5% by weight based on the total composition, which process comprises the steps of obtaining a container comprising said liquid composition, shaking said container by hand, prior to consumption. For e.g. convenience, it is preferred that the shaking of the container is performed on the still closed container.

The invention further relates to the use of the compositions as set out herein as a skin care by ingestion product. By a skin care by ingestion product is to be understood a product that is designed, offered or advertised as to be beneficial for skin care or appearance when the product is ingested, preferably regularly, and preferably by women, and most preferably by post-menopausal women. By beneficial for skin care or appearance is meant herein a product that may be able (upon prolonged use, e.g. for at least 6 months, taken at least 5 times a week) to reduce (the visibility of) fine lines and/or wrinkles and/or age spots.

### EXAMPLES

### Examples 1-4

A series of emulsions were prepared with the composition as in table 1 below.

TABLE 1

| | Protein | Estimated | | | | |
|---|---|---|---|---|---|---|
| | content | amount of β- | | | | |
| Ingredient | WPI | lactoglobulin | Example 1 | Example 2 | Example 3 | Example 4 |
| WPI 1 | 88% | 0.33% | 0.50% | | | |
| WPI 2 | 90% | 0.35% | | 0.52% | | |
| WPI 3 | 73-78% | 0.33-0.35% | | | 0.60% | |
| WPI 4 | 92% | 0.33% | | | | 0.48% |
| Water | | | balance | balance | balance | balance |
| Sunflower oil | | | 2.9% | 2.9% | 2.9% | 2.9% |
| Isoflavones | | | 0.1% | 0.1% | 0.1% | 0.1% |
| Vitamin E | | | 0.06% | 0.06% | 0.06% | 0.06% |
| Pectin (2% in | | | 20% | 20% | 20% | 20% |
| aqueous slurry | | | | | | |
| with sucrose) | | | | | | |
| Sucrose | | | 1.2% | 1.2% | 1.2% | 1.2% |
| Lycopene | | | 0.05% | 0.05% | 0.05% | 0.05% |
| apple juice | | | 13.3% | 13.3% | 13.3% | 13.3% |
| concentrate | | | | | | |
| Vitamin C | | | 0.36% | 0.36% | 0.36% | 0.36% |
| (ascorbic acid) | | | | | | |
| | | | 100% | 100% | 100% | 100% |

For examples 1-4, 4 different commercial whey protein isolates (WPI) were used as ingredient to keep the isoflavones fluffy enough to be sufficiently shakable, of which the amount in the examples 1-4 is given in the 4 right-hand columns). Each WPI has a different whey protein content, as specified by the manufacturer, from which the amount of β-lactoglobulin present in the composition was estimated, assuming that the level of β-lactoglobulin in whey protein is about 75% of the total whey proteins.

WPI 1 was: Lacprodan DI-9224 (Arla). WPI 2 was: Lacprodan DI-9213 (Arla). WPI 3 was: Nutrilac QU-7560 (Arla). WPI 4 was: WPI 895 (Fronterra). Lactose levels were: 0.2%, 0.2%, 7%, 0.5%, for WPI 1, 2, 3 and 4, respectively.

Isoflavone was Novasoy 700 (ex ADM). Lycopene was lycopene powder: Tomat-O-Red 10% CWD (cold water dispersible) (ex Lycored).

Part of the sunflower oil was used to dose and disperse the isoflavones, the remaining was to have a model for isoflavone stabilisation in O/W emulsions containing about 2-4% of oil phase. The pectin (HM) was used as a 2% in sucrose composition (i.e. concentration pectin in liquid composition is about 0.4%). The pH was about 3.7 (due e.g. to vitamin C and apple juice concentrate).

These examples were all prepared by the same process, as set out below:
the whey protein isolate was mixed with half the water (demi-water) of 45° C.,
pectin and part of the sucrose were mixed with the other half of the water (demi-water) of 80° C.,
the above two preparations were mixed, and to that was added vitamin E (in some of the sunflower oil) and mixed,
to that was added apple juice concentrate and vitamin C and mixed,
to that was added lycopene (from tomato, containing 10% pure lycopene), remaining sucrose, isoflavones and mixed,
to that was added the remainder of the sunflower oil (about 70% of the original amount),
the mixture was homogenised (high pressure homogeniser Niro Soavi Panda 2K, two stages, 200 bar first stage, 50 bar second stage, with a flow of about 25-30 litres/hour),
the mixture was pasteurised for 8 seconds at 105° C.,
the so-obtained emulsion was filled in transparent bottles of 120 ml, filled for about 80%.

### Results Examples 1-4

The so-prepared emulsions were stored both at 5° C. and at 25° C., and evaluated after 1 month and after 6 weeks. All samples showed after 1 month a sediment to be formed on the bottom. After shaking (each shaking operation done by hand, but as much as possible standardised, 10 movements up and down—up and down together counting as one movement—, amplitude about 10 cm).

TABLE 2

| Example | 5° C., 1 month | 5° C., 6 weeks |
|---|---|---|
| 1 | shakeable, no sediment remained | shakeable, no sediment |
| | | remained |
| 2 | shakeable, no sediment remained | shakeable, no sediment |
| | | remained |
| 3 | shakeable, no sediment remained | shakeable, some sediment |
| | | remained |
| 4 | shakeable, no sediment remained | shakeable, no sediment |
| | | remained |
| Example | 25° C., 1 month | 25° C., 6 weeks |
| 1 | shakeable, no sediment remained | shakeable, no sediment |
| | | remained |
| 2 | shakeable, no sediment remained | shakeable, no sediment |
| | | remained |
| 3 | shakeable, no sediment remained | shakeable, some sediment |
| | | remained |
| 4 | shakeable, no sediment remained | shakeable, no sediment |
| | | remained |

### Examples 5-6

Two emulsions were prepared with the composition as in table 3 below, having a pH of about 4.

TABLE 3

| Ingredient | Example 5 (control) | Example 6 |
|---|---|---|
| Soy protein isolate | 0.5% | |
| WPI | | 0.5% |
| Water | balance | balance |
| Fish oil | 2.7% | 2.7% |
| Isoflavones | 0.1% | 0.1% |
| Sucrose | 1.0% | 1.0% |
| apple juice concentrate | 13.3% | 13.3% |
| K-citrate | 0.2% | 0.2% |
| Vitamin E | 0.06% | 0.06% |
| Pectin slurry | 20% | 20% |
| Lycopene | 0.05% | 0.05% |
| Vitamin C (ascorbic acid) | 0.36% | 0.36% |
| | 100% | 100% |

For examples 5-6, a whey protein isolate (i.e. containing β-lactoglobulin) was compared in its stabilising power to a soy protein isolate as ingredient to keep the isoflavones fluffy enough to be sufficiently shakable. These examples were all prepared by the same process as set out below:
the protein source was mixed with half the water (demi-water) of 45° C.,
pectin and half the sucrose were mixed with the other half of the water (demi-water) of 80° C.,
fish oil, vitamin E and isoflavones were added to the WPI mixture and homogenised (2-stage high pressure homogeniser, 200 bar, 50 bar),
the pectin/sucrose slurry was added thereto and mixed,
lycopene and remaining sucrose were added thereto and mixed,
fruit concentrate, vitamin C and potassium citrate was added thereto and mixed,
the resulting product was homogenised (2-stage high pressure homogeniser, 200 bar, 50 bar),
pasteurised and filled in the same way as in example 1.

The so-prepared emulsions were stored at 5° C., and evaluated after 1 month. Both samples showed after 1 month a sediment had formed on the bottom. The result after shaking is set out in the table below. Similar shaking as in examples 1-4.

TABLE 4
results examples 5-6

| Example | 5° C., 1 month |
|---|---|
| 5 | sediment, not shakeable |
| 6 | shakeable, no sediment remained |

### Examples 7-8

A series of emulsions were prepared with the composition as in the table below, using two different processing options.

TABLE 5

| Ingredient | Example 7 | Example 8 |
|---|---|---|
| WPI | 0.4% | 0.4% |
| Water | balance | balance |
| Fish oil concentrate | 3.0% | 2.7% |
| Sunflower oil | 0 | 0.32 |
| Isoflavones | 0.1% | 0.1% |
| Vitamin E | 1.68% | 1.68% |
| Pectin (2% in slurry with sucrose) | 20% | 20% |
| Sucrose | 1.2% | 1.7% |
| Lycopene | 0.05% | 0.05% |
| apple juice concentrate | 13.3% | 13.3% |
| Vitamin C | 0.36% | 0.36% |
| | 100% | 100% |

Examples 7 and 8 were made following the process as for examples 5 and 6, except that for example 7 the first homogenisation step is omitted.

The so-prepared emulsions were stored both at 5° C., and evaluated after 2 weeks. The samples showed after 1 month a sediment to had formed on the bottom. After shaking (each shaking operation done by hand, but as much as possible standardised, 10 movements up and down, amplitude about 10 cm).

TABLE 6
results examples 7-8

| Example | 5° C., 2 weeks |
|---|---|
| 7 | shakeable, no sediment remained |
| 8 | shakeable, no sediment remained |

### Examples 9, 10

The following products were prepared, using processing as below in table 7:

TABLE 7

| Ingredient | Example 9 | Example 10 |
|---|---|---|
| Whey protein isolate | 0.5% | 0.5% |
| Water | Balance | Balance |
| Fish oil concentrate | 3.0% | 2.9% |
| Sunflower oil | — | 0.4% |
| Isoflavones | 0.1% | 0.1% |
| Vitamin E | 0.1% | 0.1% |
| Pectin powder | 0.4% | 0.4% |
| Sucrose | 1.0% | 2.0% |
| Lyc-O-Mato Red 0.15% paste | 3.3% | — |
| Tomat-O-Red 10% CWD powder | — | 0.05 |
| Grape juice concentrate | 16% | 16.5% |
| Vitamin C (ascorbic acid) | 0.3% | 0.3% |
| Citric acid | — | 0.1 |
| | 100% | 100% |

The whey protein isolate was Lacprodan DI-9213.
The Lyc-O-Mato Red 0.15% paste was a high-lycopene tomato paste ex Lycored.
The Tomat-O-Red 10% CWD powder was a high lycopene tomato powder ex Lycored.
Pectin powder was HM pectin AMD 880 ex Danisco.

### Processing Example 9.

the pectin was hydrated in about 98% of the (demi-)water at 60° C., and the whey protein isolate was added thereto and mixed until hydrated,
isoflavone and sucrose were added thereto and mixed,
tomato-paste (tomato powder for example 10) was added thereto and mixed,
grape juice and vitamin C (in remaining 2% of the water) were added thereto,
fish oil containing the vitamin E were added thereto and mixed,
the mixture was homogenised (Gaulin high pressure homogeniser, two stages, 200 bar first stage, 50 bar second stage),
the product was pasteurised at 105° C. for 8 seconds,
the so-obtained emulsion was aseptically filled in aluminium bottles of 66 ml with a headspace of about 10%,
after 4 weeks storage at 5° C. the viscosity was measured as described below.

### Processing Example 10.

whey protein hydrolysate was hydrated in about 70% of the (demi-)water at 45° C.
pectin and part of the sucrose was hydrated in about 20% of the (demi-)water at 80° C.
a blend of sunflower oil, vitamin E and isoflavones was added to hydrated to the hydrated whey protein hydrolysate
to this was added the pectin/sucrose slurry and stirred
to this lycopene and remaining sucrose were added and stirred
to this was added a solution of citric acid and vitamin C in the remaining water and grape juice concentrate,
to this was added the fish oil
the mixture was homogenised (Gaulin high pressure homogeniser, two stages, 200 bar first stage, 50 bar second stage),
the product was pasteurised at 105° C. for 8 seconds,
the so-obtained emulsion was aseptically filled in aluminium bottles of 66 ml, with a headspace of about 10%
after 4 weeks storage at 5° C. the viscosity was measured as described below.

The viscosity of the products was measured using the following equipment: Advanced Rheometer AR 1000, spindle: conic, 2°0′38″, diameter (mm) 60, truncation (microm) 59, shear rate of about 10 (s⁻¹) (9.06 s⁻¹ in fact) at 7° C. Example 9 had a viscosity of about 0.09 Pa·s, and a pH of 3.6 and example 10 had a viscosity of about 0.02 Pa·s and a pH of about 3.3.

### Example 11

Model samples of isoflavones and acid whey protein isolate only in water were prepared. Levels of Novasoy 700 (ex ADM, containing a level of isoflavones of 70%) were: 0.075%, 0.105%, and 0.135% (all weight % on total formulation). Levels of whey protein isolate (Lacprodan DI-9213 ex Arla, see example 1 for β-lactoglobulin content) were: 0.3%, 0.5%, 0.7% and 0.9% (all weight % based on the total formulation). Samples were prepared by dissolving the protein in demineralised water at 65° C. in a beaker with a turrax. After 5 minutes the isoflavones were mixed in, and mixing continued for about 5 minutes. The so-obtained emulsion was filled in transparent bottles of 180 ml, filled for about 80% of its volume. Products were stored at 5° C., and re-dispersibility by shaking was checked for the samples after 3 days and after 21 days storage. The results are set out below, in which tables 8 and 9 it was estimated what amount of isoflavones could be re-dispersed after shaking in the same way as in example 1, judging by estimating the amount of remaining isoflavones at the bottom of the bottle.

TABLE 8
redispersibility (in % of amount of Novasoy 700) after 3 days storage
at 5° C.

| | WPI | | | |
|---|---|---|---|---|
| Novasoy 700 | 0.3 (wt %) | 0.5 (wt %) | 0.7 (wt %) | 0.9 (wt %) |
| 0.075 (wt %) | 80-90% | 90-100% | 90-100% | 90-100% |
| 0.105 (wt %) | 80-90% | 90-100% | 90-100% | 90-100% |
| 0.135 (wt %) | 80-90% | 90-100% | 90-100% | 90-100% |

TABLE 9
redispersibility (in % of amount of Novasoy 700) after 21 days storage
at 5° C.

| | WPI | | | |
|---|---|---|---|---|
| Novasoy 700 | 0.3 (wt %) | 0.5 (wt %) | 0.7 (wt %) | 0.9 (wt %) |
| 0.075 (wt %) | 90-100% | 90-100% | 90-100% | 90-100% |
| 0.105 (wt %) | 80-90% | 90-100% | 90-100% | 90-100% |
| 0.135 (wt %) | 80-90% | 80-90% | 90-100% | 90-100% |

1. Liquid composition comprising:
isoflavones in an amount of 0.005-0.5%, by weight, based on the total formulation,
water in an amount of 40-98%, by weight, based on the total formulation,
β-lactoglobulin in an amount of 0.1-2%, by weight on the total formulation,
wherein the composition comprises casein in amount less than 0.5%, by weight based on the total composition, and wherein the weight ratio β-lactoglobulin to isoflavone is between 2 and 30.

2. Liquid composition according to claim 1, wherein the amount of β-lactoglobulin in the composition is 0.1-1%, preferably 0.15-0.5%, more preferably 0.25-0.45% by weight on the total formulation.

3. Liquid composition according to claim 1 or 2, wherein the β-lactoglobulin originates from whey protein isolate, which is present in the composition in an amount of 0.1-2%, preferably 0.2-1.5% by weight, based on the total composition.

4. Liquid composition according to any of claims 1 to 3, wherein the amount of isoflavone is 0.01-0.2%, preferably 0.02-0.1%, by weight based on the total composition.

5. Liquid composition according to any of claims 1 to 4, wherein the weight ratio β-lactoglobulin to isoflavone is between 2 and 15, preferably between 3 and 10.

6. Liquid composition according to any of claims 1 to 5, wherein β-lactoglobulin is present as whey protein isolate, and the (weight) ratio whey protein isolate: isoflavone is between 1.5 and 40, preferably between 3 and 22, more preferably between 4.5 and 15.

7. Liquid composition according to any of claims 1 to 6, further comprising 0.2-10% of a hydrophobic matter.

8. Liquid composition according to any of claims 1 to 7, further comprising a charged carbohydrate-based biopolymer in an amount of 0.1-1.2%, more preferably in an amount of 0.2-0.6% by weight, based on the total composition

9. Liquid composition according to any of claims 1 to 8, wherein the product has a pH of below 5, preferably 2.8-4.5, more preferably 3-4.

10. Liquid composition according to any of claims 1 to 9, wherein the composition comprises casein in amount less than 0.2%, by weight based on the total composition, preferably less than 0.1%.

11. Liquid composition according to any of claims 1 to 10, wherein the ratio casein or caseinate to β-lactoglobulin is below 2, preferably below 1.

12. Liquid composition according to any of claims 1 to 11, characterised in that the composition has a viscosity of 0.005-0.2 Pa·s, preferably 0.01-0.15 Pa·s, more preferably 0.01-0.1 Pa·s when measured at a shear rate of 10 (s⁻¹) at a temperature of 7° C. using as equipment an Advanced Rheometer AR 1000, spindle: conic, 2°0′38″, diameter (mm) 60, truncation (microm) 59.

13. Process for preparing a liquid composition comprising isoflavones in an amount of 0.005-0.5% by weight based on the total formulation, water in an amount of 40-98% by weight based on the total formulation, β-lactoglobulin in an amount of 0.1-2% by weight on the total formulation, casein in amount less than 0.5%, by weight based on the total composition, and wherein the weight ratio β-lactoglobulin to isoflavone is between 2 and 30, which process comprises the steps of:
a) preparing a mixture comprising the water, isoflavones and β-lactoglobulin,
b) vigorous stirring said mixture,
c) subjecting to a pasteurising or sterilising treatment and packing, in any given order.

14. Process according to claim 13, wherein the water has a temperature of 30-60° C. when the β-lactoglobulin is added to the aqueous phase.

15. Process according to any of claim 13 or 14, characterised by adding omega-3 fatty acids and/or omega-6 fatty acids and/or esters thereof during any step prior to step c), and wherein the vigorous stirring comprises homogenisation.

16. Process according to any of claims 13 to 15, wherein at any step before step c) is added one or more of a charged biopolymer, pH reducing agents, flavourings, carotenoids, vitamins, minerals, flavouring agents, colouring agents, vegetable oil.

17. Process for preparing a liquid composition ready for consumption, said liquid composition comprising isoflavones in an amount of 0.005-0.5% by weight, based on the total formulation, water in an amount of 40-98% by weight, based on the total formulation, β-lactoglobulin in an amount of 0.1-2% by weight on the total formulation, casein in an amount of less than 0.5% by weight, based on the total formulation, and wherein the weight ratio β-lactoglobulin to isoflavone is between 2 and 30, which process comprises the steps of obtaining a container comprising said liquid composition, shaking said container by hand, prior to consumption.

18. Use of composition according to any of claims 1 to 12, as a skin care by ingestion product.

19. Use according to claim 18, by women, preferably by post-menopausal women.

20. Use according to claim 18 or 19, to reduce (the visibility of) fine lines and/or wrinkles and/or age spots.
